(19) United States
(12) Patent Application Publication
(10) Pub. No.: US 2023/0019651 A1
(43) Pub. Date: Jan. 19, 2023

(54) UV LIGHT STERILIZATION DEVICE

(71) Applicant: CHEN-SOURCE INC., Taoyuan (TW)

(72) Inventors: Shui-Yuan WANG, Taoyuan (TW); Kuan-Yen WU, Taoyuan (TW)

(21) Appl. No.: 17/375,675

(22) Filed: Jul. 14, 2021

(51) Int. Cl. A61L 2/10

(52) U.S. Cl. CPC A61L 2/10; A61L 2202/11; A61L 2202/122; A61L 2202/14

(57) ABSTRACT

A UV light sterilization device includes a main body with an accommodation space defined therein for a predetermined item to be placed in a horizontal direction, a power supply module mounted in one side plate of the main body and providing an electrical connection port, and a UV lamp set with a drive control circuit assembled on the bottom side of the top cover of the main body and electrically connected to the electrical connection port, the drive control circuit having a plurality of evenly distributed UV LED chips for downwardly irradiating the surface of the predetermined item that is placed in the main body.

### BACKGROUND OF THE INVENTION

### 1. Field of the Invention

The present invention relates to sterilization device technology and more particularly, to a UV light sterilization device, which is to be placed on a predetermined plane, and then make the flip cover of the UV light sterilization device open in a rotating manner, and place the predetermined item in the accommodation space of the UV light sterilization device in the horizontal direction, then close the flip cover, and press the start key to perform disinfection and sterilization which lasts for nearly 900 seconds. The start key is an indicator button switch, which can display all states through different colors or different flashing lights. When the user accidentally opens the flip cover during the sterilization operation, the door sensor switch that is located inside the UV lamp set can sense the state of the flip cover, and the start key is turned off through the switch of the power relay to instantly turn off the power supply to multiple UV LED chips to form a protection mechanism for the user's eyes and at the same time take into account the convenience of use and portability.

### 2. Description of the Related Art

With the rapid development of electronic technology and multimedia information, electronic devices such as smart phones, tablet computers, and notebook computers are developing toward light, thin, short, small, and powerful trends, mainly for small size, light weight, and convenient portability. Coupled with continuous innovation of soft and hardware functions, it is more flexible and practical in use. In the process of using the aforementioned electronic products, it is easy to cause the sticky grease of yourself or others, the droplets produced by the oral cavity and the related pathogens constituted by bacteria and viruses to remain on the surface of the aforementioned electronic products such as tablet computers or notebook computers. Even if the user has done hand cleaning or disinfection later, it is easy to return the remaining pathogens to the hand when the user takes it again or touches it with his hand. After the hands with pathogens come into contact with mucosal organs such as eyes, nose, mouth, etc., it will cause serious harm to human health or life. For example, some of the infection pathways of the recent global epidemic of new coronary pneumonia (COVID-19) are generated through the aforementioned channels. The traditional method of spraying or wiping with detergent or alcohol can achieve the functions of disinfection and sterilization, but if the user's hands accidentally come into contact with the detergent or alcohol, it is easy to cause skin irritation or chemical medicine remains, and after a while, it is difficult to reliably perform disinfection and sterilization actions due to negligence or fatigue.

Therefore, in an era when many bacteria or viruses are spreading in the environment due to climate change and globalization, how the industry can design a device that can sterilize electronic devices in a plate shape, while taking into account the convenience and portability of use, is an important issue that those engaged in this industry want to improve.

### SUMMARY OF THE INVENTION

The present invention has been accomplished under the circumstances in view. It is the main object of the present invention to provide a UV light sterilization device, which comprises a main body, a power supply module and a UV lamp set. The main body comprises a top cover, two side plates respectively provided at two opposite lateral sides of the top cover, a flip cover pivotally connected to one side of the top cover between the two side plates and turnable upwardly relative to the top cover, and an accommodation space surrounded by the top cover, the side plates and the flip cover for a predetermined item to be placed in a horizontal direction. The power supply module is mounted in one side plate, comprising an electrical connection port mounted in the other side plate. The UV lamp set is assembled on the bottom side of the top cover inside the accommodation space, comprising a drive control circuit electrically connected to the electrical connection port. The drive control circuit comprises a plurality of evenly distributed UV LED chips for downwardly irradiating the surface of the predetermined item that is placed in the accommodation space. By the aforementioned composition, place the UV light sterilization device on a predetermined plane, and make the flip cover open in a rotating manner, and place the predetermined item in the accommodation space in the horizontal direction, then close the flip cover, and press the start key protruding on the side plate to perform disinfection and sterilization which lasts for nearly 900 seconds. The start key is an indicator button switch, which can display all states through different colors or different flashing lights. When the user accidentally opens the flip cover during the sterilization operation, the door sensor switch that is located inside the UV lamp set can sense the state of the flip cover, and the start key is turned off through the switch of the power relay to instantly turn off the power supply to multiple UV LED chips to form a protection mechanism for the user's eyes and at the same time take into account the convenience of use and portability.

It is another object of the present invention to provide a UV light sterilization device, wherein the power supply module further comprises a power input unit for inputting power provided by an external power supply, a first control board electrically connected to the power input unit, and a first microcontroller provided on the first control board.

It is still another object of the present invention to provide a UV light sterilization device, wherein the UV lamp set further comprises a starting unit. The starting unit comprises a start key and at least one door sensor switch protruding from one side plate. The drive control circuit further comprises a second control board and an electronic ballast. The second control board comprises a second microcontroller and a power relay. The electronic ballast is set between the power relay and the UV LED chips. The start key comprises a first contact, a second contact, a third contact and a fourth contact. The first contact and the fourth contact are electrically connected to the second control board. The second contact and the third contact are electrically connected to the second microcontroller. After the start key and the door sensor switch are connected in parallel, the start key is electrically connected to the second control board to form a start detection loop. The power relay has an on and off state to switch between the start key and the door sensor switch.

It is still another object of the present invention to provide a UV light sterilization device, wherein the start key is selectively composed of an indicator button switch or a rotary switch. The indicator button switch is a transparent button cover with at least one light emitting diode inside.

It is still another object of the present invention to provide a UV light sterilization device, wherein the door sensor switch is selectively composed of a mechanical button switch or an electronic infrared sensor switch.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is an oblique top elevational view of a UV light sterilization device in accordance with the present invention.

FIG. 2 is an oblique bottom elevational view of the UV light sterilization device in accordance with the present invention.

FIG. 3 is an exploded view of the UV light sterilization device and a storage box.

FIG. 4 is an oblique top elevational view of the storage box.

FIG. 5 is a functional block diagram of the power supply module and UV lamp set inside the UV light sterilization device of the present invention.

FIG. 6 is a schematic diagram of the operation of the UV light sterilization device of the present invention.

### DETAILED DESCRIPTION OF THE PREFERRED EMBODIMENT

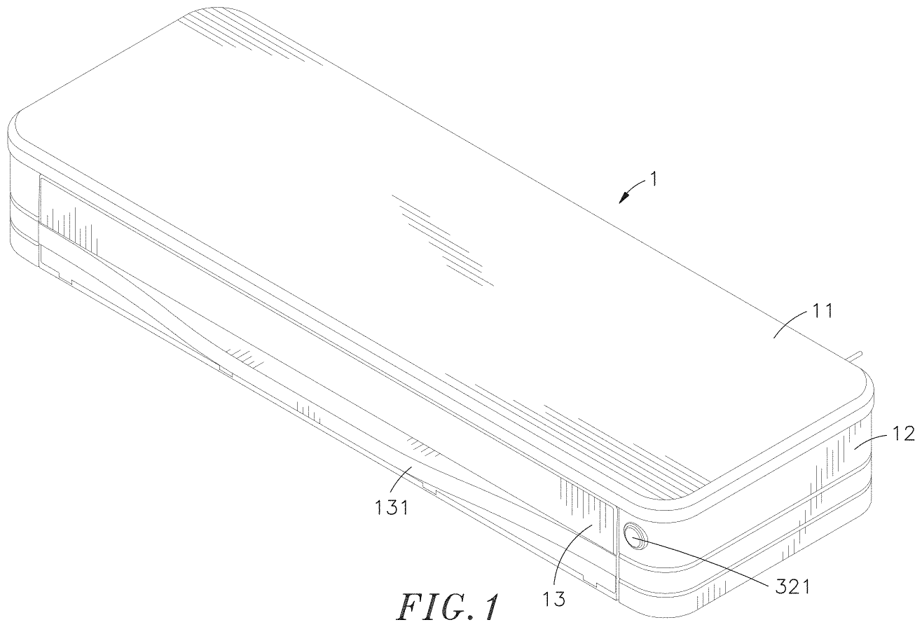

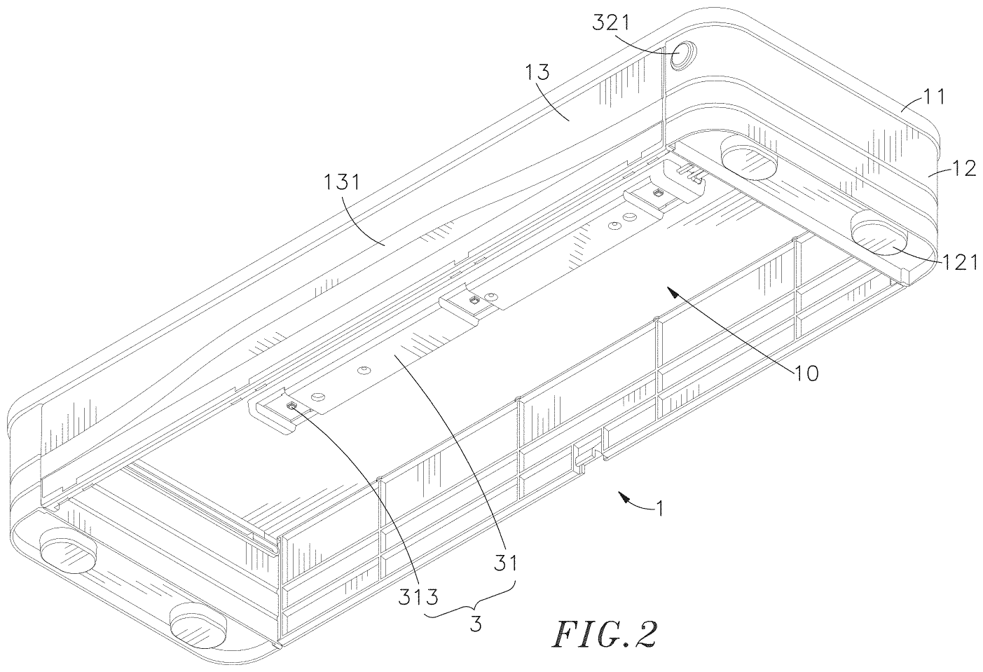

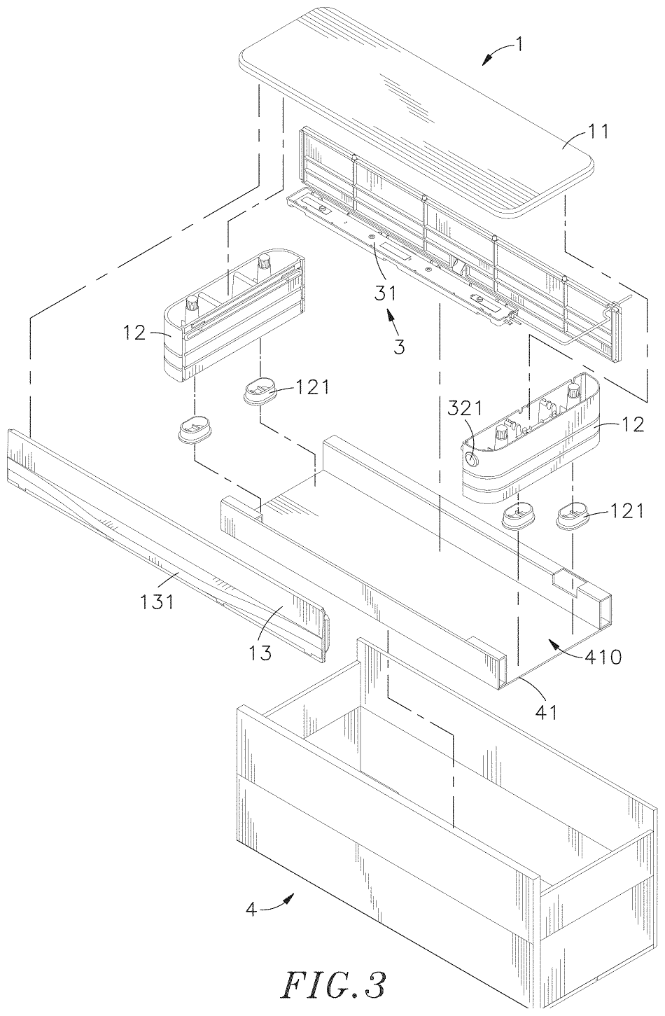

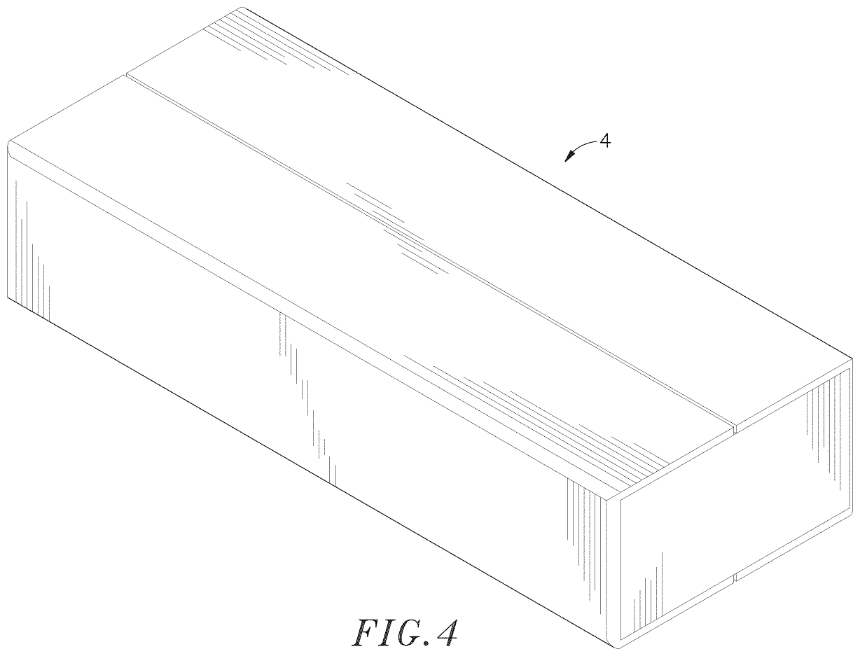

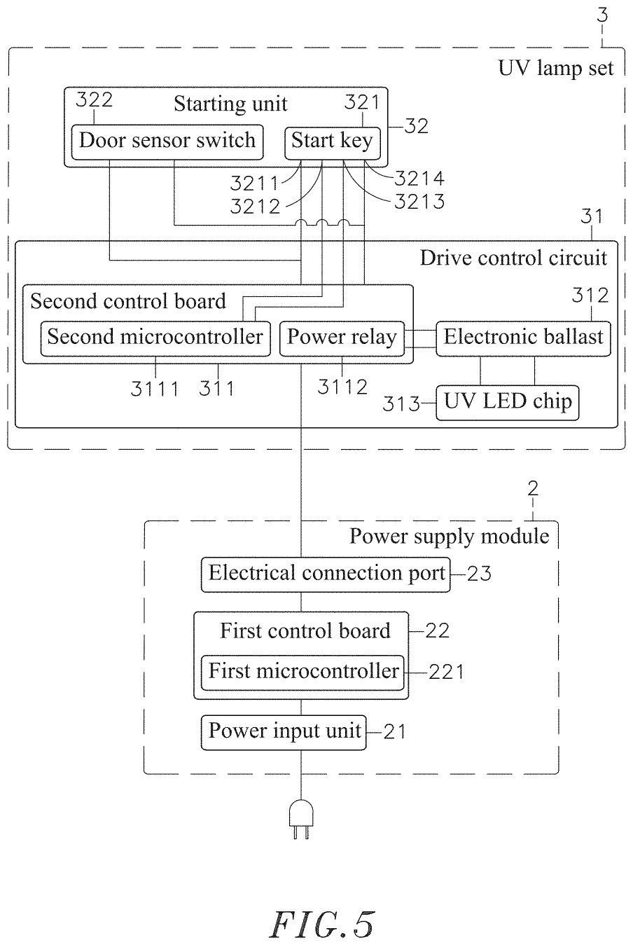

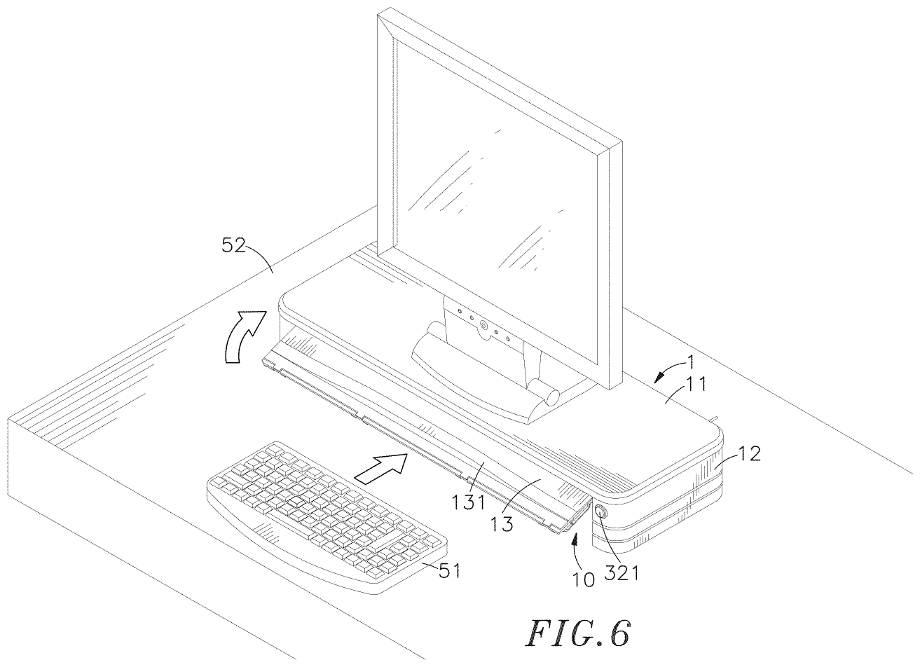

Please refer to FIGS. 1-6, wherein FIG. 1 is an oblique top elevational view of a UV light sterilization device in accordance with the present invention; FIG. 2 is an oblique bottom elevational view of the UV light sterilization device; FIG. 3 is an exploded view of the UV light sterilization device and a storage box; FIG. 4 is an oblique top elevational view of the storage box; FIG. 5 is a functional block diagram of the power supply module and UV lamp set inside the UV light sterilization device of the present invention; FIG. 6 is a schematic diagram of the operation of the UV light sterilization device of the present invention. The UV light sterilization device of the invention comprises a main body 1, a power supply module 2 and a UV lamp set 3.

The main body 1 has a top cover 11 above and a side plate 12 on each of two opposite lateral sides thereof. The power supply module 2 is provided inside one of the two side plates 12, and an electrical connection port 23 is provided on the other side of the power supply module 2. At least one side between the two side plates 12 is provided with a flip cover 13 that can be turned upwards. The flip cover 13 is further provided with a force applying block 131 for applying force and turning upward. The main body 1 is further concavely provided with an accommodation space 10 for a predetermined item 51 to be placed in a horizontal direction.

The UV lamp set 3 is assembled on a bottom side of the top cover 11. The UV lamp set 3 comprises a drive control circuit 31 that is electrically connected to the electrical connection port 23. The drive control circuit 31 comprises a plurality of UV LED chips 313 that can be evenly distributed and irradiate the surface of the predetermined item 51 downward. These UV LED chips 313 can emit ultraviolet wavelength range between 260˜280 nm.

The bottoms of the two side plates 12 are each protrudingly provided with at least one pad 121 that resists a predetermined plane, and the pads 121 are made of rubber, silicone or plastic material with a high friction coefficient.

The power supply module 2 comprises a power input unit 21 for inputting power provided by an external power supply. The power supply module 2 further comprises a first control board 22 connected to the power input unit 21 and a first microcontroller 221 is provided on the first control board 22.

The above-mentioned UV lamp set 3 further comprises a starting unit 32. The starting unit 32 comprises a start key 321 and at least one door sensor switch 322 protruding from the side plate 12. The drive control circuit 31 further comprises a second control board 311 and an electronic ballast 312. The second control board 311 has installed thereon a second microcontroller 3111 and a power relay 3112. The electronic ballast 312 is set between the power relay 3112 and the UV LED chips 313. The first contact 3211 and the fourth contact 3214 in the start key 321 are connected to the second control board 311, and the second contact 3212 and the third contact 3213 are connected to the second microcontroller 3111. After the start key 321 and the door sensor switch 322 are connected in parallel, the start key 321 is electrically connected to the second control board 311 to form a start detection loop. The power relay 3112 has an on and off state to switch between the start key 321 and the door sensor switch 322. The start key 321 is composed of an indicator button switch (not shown) or a rotary switch (not shown). The indicator button switch is a transparent button cover (not shown) with at least one light emitting diode (not shown) inside. The door sensor switch 322 is composed of a mechanical button switch (not shown) or an electronic infrared sensor switch (not shown).

The UV light sterilization device can be accommodated in a storage box 4. The storage box 4 has a positioning seat 41 placed therein. The positioning seat 41 is provided with a positioning space 410 for positioning the UV light sterilization device.

The predetermined item 51 refers to a computer keyboard, a notebook, a tablet PC, an e-book reader or a smartphone.

In actual operation, the UV light sterilization device of the present invention is first taken out from the positioning seat 41 of the storage box 4 and placed on a predetermined plane 52. The predetermined plane 52 can be an opaque flat table, and the opaque table can avoid unnecessary reflections when UV light is irradiated, so as to ensure that every part of the predetermined item 51 can be disinfected by UV light normally. After the UV light sterilization device is positioned on the predetermined plane 52, you can insert a USB cable to the power supply module 2 and use the city power supply to supply power. Of course, those who are familiar with this art can also install a rechargeable battery (such as lithium battery) and a wired or wireless charging module inside the power supply module 2 to form a portable UV light sterilization device, which can be free from cables to improve the convenience of use. During operation, the flip cover 13 on the side of the main body 1 is opened in a rotating manner, and the predetermined item 51 is placed in the accommodation space 10 in the horizontal direction, and then the flip cover 13 is closed, and then press the start key 321 to perform disinfection and sterilization operations that lasted nearly 900 seconds. If the user feels that the 900-second disinfection operation time is insufficient, the start key 321 can be pressed again to perform another 900-second disinfection operation time without opening the flip cover 13, and the flip cover 13 can be opened and the predetermined item 51 can be taken out until the user's requirements are met. The start key 321 is an indicator button switch, which can display all states through different colors or different flashing lights. For example: when the start key 321 is blue and constantly lit, it means that the UV light sterilization device is in the power-on state; when the start key 321 is red and constantly lit, it means that the predetermined item 51 has been placed inside the UV light sterilization device and is in a waiting state; when the start key 321 is blue and flashing, it means that the UV light sterilization device is being disinfected. Of course, the light signal of the aforementioned start key 321 shows that the various states of the UV light sterilization device are only one of a plurality of embodiments, and the present invention is not limited by this. For example, all implementations in which the status is displayed by the start key 321 through any color or any form of flashing light signal are within the protection scope of the present invention.

When the user turns on the flip cover 13 by mistake during the above-mentioned sterilization operation, the door sensor switch 322 installed in the UV lamp set 3 can sense the status of the flip cover, and the start key 321 is turned off through the switch of the power relay 3112. A contact and B contact are switched between the start key 321 and the door sensor switch 322 through the power relay 3112 magnetic absorption to present the interleaved state of short-circuit open and open-circuit closed, so the power supply to the multiple UV LED chips 313 can be turned off immediately to form a protection mechanism for the user's eyes.

Please refer to FIG. 6, in a preferred embodiment, the UV light sterilization device can be placed on a work desk, and a computer screen is placed on the top cover 11 (the UV light sterilization device acts as a screen stand), and the predetermined item 51 is a computer keyboard. After the user has operated the computer keyboard for a period of time, the surface of the plural keys is stained with adhesive grease, oral droplets, bacteria, and viruses that constitute related pathogens. When the work comes to an end, the user opens the flip cover 13 of the UV light sterilization device and pushes the computer keyboard into its accommodation space 10, then closes the flip cover 13 and then presses the start key 321 for disinfection and sterilization. When the user waits for 15 minutes, the computer keyboard can be taken out, and the next stage of work can be carried out to form a kind of daily operation UV light sterilization device. The convenience of use can promote the sales of the UV light sterilization device in the market.

According to the disclosure in FIGS. 1 to 6 above, it can be understood that the present invention is a UV light sterilization device, which comprises a main body that comprises a top cover, two side plates respectively provided at two opposite lateral sides of the top cover, a flip cover pivotally connected to one side of the top cover between the two side plates and turnable upwardly relative to the top cover and an accommodation space surrounded by the top cover, the side plates and the flip cover for a predetermined item to be placed in a horizontal direction; a power supply module mounted in one side plate and comprising an electrical connection port mounted in the other side plate; and a UV lamp set assembled on the bottom side of the top cover inside the accommodation space and comprising a drive control circuit that is electrically connected to the electrical connection port and comprises a plurality of evenly distributed UV LED chips for downwardly irradiating the surface of the predetermined item that is placed in the accommodation space. By the aforementioned composition, place the UV light sterilization device on a predetermined plane, and make the flip cover open in a rotating manner, and place the predetermined item in the accommodation space in the horizontal direction, then close the flip cover, and press the start key protruding on the side plate to perform disinfection and sterilization which lasts for nearly 900 seconds. The start key is an indicator button switch, which can display all states through different colors or different flashing lights. When the user accidentally opens the flip cover during the sterilization operation, the door sensor switch that is located inside the UV lamp set can sense the state of the flip cover, and the start key is turned off through the switch of the power relay to instantly turn off the power supply to multiple UV LED chips to form a protection mechanism for the user's eyes and at the same time take into account the convenience of use and portability. The invention is used in the field of disinfection and sterilization of mobile electronic devices or computer peripherals, and has excellent practicability, so a patent application was filed to seek protection of the patent right.

Although a particular embodiment of the invention has been described in detail for purposes of illustration, various modifications and enhancements may be made without departing from the spirit and scope of the invention. Accordingly, the invention is not to be limited except as by the appended claims.

What the invention claimed is:

1. A UV light sterilization device, comprising:
a main body, said main body comprising a top cover, two side plates respectively provided at two opposite lateral sides of said top cover, a flip cover pivotally connected to one side of said top cover between said two side plates and turnable upwardly relative to said top cover, and an accommodation space surrounded by said top cover, said side plates and said flip cover for a predetermined item to be placed in a horizontal direction;
a power supply module mounted in one said side plate, said power supply module comprising an electrical connection port mounted in the other said side plate; and
a UV lamp set assembled on a bottom side of said top cover inside said accommodation space, said UV lamp set comprising a drive control circuit electrically connected to said electrical connection port, said drive control circuit comprising a plurality of evenly distributed UV LED chips for downwardly irradiating the surface of said predetermined item that is placed in said accommodation space.

2. The UV light sterilization device as claimed in claim 1, wherein said power supply module further comprises a power input unit for inputting power provided by an external power supply, a first control board electrically connected to said power input unit and a first microcontroller provided on said first control board.

3. The UV light sterilization device as claimed in claim 1, wherein said UV lamp set further comprises a starting unit, said starting unit comprising a start key and at least one door sensor switch protruding from one said side plate; said drive control circuit further comprises a second control board and an electronic ballast, said second control board comprising a second microcontroller and a power relay, said electronic ballast being set between said power relay and said UV LED chips, said start key comprising a first contact, a second contact, a third contact and a fourth contact, said first contact and said fourth contact being electrically connected to said second control board, said second contact and said third contact being electrically connected to said second microcontroller, said start key and said door sensor switch being connected in parallel, said start key being electrically connected to said second control board to form a start detection loop, said power relay having an on and off state to switch between said start key and said door sensor switch.

4. The UV light sterilization device as claimed in claim 3, wherein said start key is selectively composed of an indicator button switch or a rotary switch, said indicator button switch being a transparent button cover with at least one light emitting diode inside.

5. The UV light sterilization device as claimed in claim 3, wherein said door sensor switch is selectively composed of a mechanical button switch or an electronic infrared sensor switch.

6. The UV light sterilization device as claimed in claim 1, wherein said predetermined item refers to a computer keyboard, a notebook, a tablet PC, an e-book reader or a smartphone.

7. The UV light sterilization device as claimed in claim 1, wherein said UV LED chips are capable of emitting ultraviolet wavelength range between 260˜280 nm.

8. The UV light sterilization device as claimed in claim 1, further comprising a storage box for accommodating said main body, said storage box comprising a positioning seat therein, said positioning seat being provided with a positioning space for positioning said main body.

9. The UV light sterilization device as claimed in claim 1, wherein said flip cover of said main body is provided with a force applying block for applying force and turning upward relative to said top cover.

10. The UV light sterilization device as claimed in claim 1, wherein each said side plate has a bottom side thereof protrudingly provided with at least one pad that resists a predetermined plane, said pads being made of rubber, silicone or plastic material with a high friction coefficient.
